(19) United States
(12) Patent Application Publication
(10) Pub. No.: US 2022/0192515 A1
(43) Pub. Date: Jun. 23, 2022

(54) FLEXIBLE BIOSENSORS AND METHODS OF USING SAME TO ESTIMATE HEART RATE

(71) Applicant: Georgia Tech Research Corporation, Atlanta, GA (US)

(72) Inventors: Woon-Hong Yeo, Atlanta, GA (US); Shinjae Kwon, Atlanta, GA (US)

(21) Appl. No.: 17/559,476

(22) Filed: Dec. 22, 2021

Related U.S. Application Data
(60) Provisional application No. 63/128,175, filed on Dec. 20, 2020.
(60) Provisional application No. 63/129,191, filed on Dec. 22, 2020.

(51) Int. Cl. A61B 5/0205; A61B 5/024; A61B 5/00

(52) U.S. Cl. CPC A61B 5/0205; A61B 5/02416; A61B 5/02438; A61B 2562/164; A61B 5/7225; A61B 2562/0219; A61B 5/6833

(57) ABSTRACT

An exemplary embodiment of the present disclosure provides a wearable flexible biosensor, comprising an electrical circuit and an elastomer. The electrical circuit can be configured to generate one or more signals indicative of a wearer's photoplethysmogram (PPG) and acceleration. The elastomer can encapsulate the electrical circuit. The elastomer can have a bottom surface configured to adhere to the skin of the wearer.

### CROSS-REFERENCE TO RELATED APPLICATIONS

This application claims the benefit of U.S. Provisional Application Ser. Nos. 63/128,175 and 63/129,191, both of which were filed on 22 Dec. 2020, and both of which are incorporated herein by reference in their entireties as if fully set forth below.

### FIELD OF THE DISCLOSURE

The various embodiments of the present disclosure relate generally to biosensors and methods of using biosensors to estimate heart rate.

### BACKGROUND

Advances in miniaturization of electronics, multi-sensor integration, and low-power wireless communication have enabled new wearable devices for fitness tracking, human-machine interaction, and ambulatory monitoring. The market of wearable devices is exponentially growing, which was estimated to be worth around $25 billion by the end of 2019. Recent works have utilized the wearable device platform as a fitness tracker by monitoring wearer's physiological signals, including photoplethysmogram (PPG), heart rate (HR), and motion activities. Among them, inertial measurement sensors have been widely used in analysis of athletic performance via an assessment of time-varying activities and movement patterns. Although these devices with enclosing wristbands provide relevant information, they have inherent problems of device rigidity, discomfort, and motion artifacts. The existing devices still rely on rigid metals with plastic packaging, which limits conformal lamination onto curvilinear skin and requires the use of a tightly worn wristband. The gap between rigid sensors and curved skin creates sliding motions upon a wearer's movements, leading to significant motion artifacts in physiological signal recording. Consequently, the aforementioned issues of current wristband devices outweigh their benefits and functionality, placing hurdles in widespread adoption and further expansion of wearable technology in athletic training and daily uses. More specifically, recent advances in wearable optical HR sensors utilized new flexible materials and fabrication methods to offer a seamless, conformal contact on skin with comfortable wearability. However, these devices still require wiring to a separate data acquisition and power supply. Some devices use a short-range, wireless communication and powering method with a bulky antenna near the device, which is not practical for athletes who have intensive physical activities. Accordingly, there is a need for improved biosensors and methods of estimating heart rate using biosensors that address one or more of disadvantages discuss above.

### BRIEF SUMMARY

An exemplary embodiment of the present disclosure provides a wearable flexible biosensor, comprising an electrical circuit and an elastomer. The electrical circuit can be configured to generate one or more signals indicative of a wearer's photoplethysmogram (PPG) and acceleration. The elastomer can encapsulate the electrical circuit. The elastomer can have a bottom surface configured to adhere to the skin of the wearer.

In any of the embodiments disclosed herein, the biosensor can further comprise at least two light emitting diodes and a photodiode. The at least two light emitting diodes can be positioned proximate the bottom surface of the elastomer and configured to direct light towards the skin of the wearer. The photodiode can be positioned proximate the bottom surface of the elastomer and configured to receive light reflected from the wearer.

In any of the embodiments disclosed herein, the biosensor can further comprise a rechargeable battery configured to provide power to the electrical circuit.

In any of the embodiments disclosed herein, the biosensor can further comprise a first polymer layer, a first copper electrical interconnect layer, a dielectric layer, a second copper electrical interconnect layer, and a second polymer layer. The first polymer layer can be positioned beneath the electrical circuit. The first copper electrical interconnect layer can be positioned beneath the first polymer layer. The dielectric layer can be positioned beneath the first copper electrical interconnect layer. The second copper electrical interconnect layer can be positioned beneath the dielectric layer. The second polymer layer can be positioned beneath the second copper electrical interconnect layer. The elastomer can encapsulate the first polymer layer, the first copper electrical interconnect layer, the first copper electrical interconnect layer, the dielectric layer, the second copper electrical interconnect layer, and the second polymer layer.

In any of the embodiments disclosed herein, the biosensor can further comprise a wireless transceiver configured to transmit the one or more signals indicative of a wearer's photoplethysmogram (PPG) and acceleration to a remote device.

In any of the embodiments disclosed herein, the elastomer can be configured to prevent water exterior to the biosensor from migrating into electrical circuit.

In any of the embodiments disclosed herein, the biosensor can be capable of bending 180 degrees with a radius of curvature of about 1.5 mm.

In any of the embodiments disclosed herein, the electrical circuit can comprise an input and an output. The biosensor can be configured such that a resistance between the input and output changes less than 1.0 ohms if the biosensor is subjected to 100 cycles of bending over a range of 0 to 180 degrees with a minimum radius of curvature of 1.5 mm.

In any of the embodiments disclosed herein, the electrical circuit can comprise an input and an output. The biosensor can be configured such that a resistance between the input and output changes less than 0.001-0.5 ohms if the biosensor is subjected to 100 cycles of bending over a range of 0 to 180 degrees with a minimum radius of curvature of 1.5 mm.

In any of the embodiments disclosed herein, the biosensor can be configured as a patch.

Another embodiment of the present disclosure provides a method of estimating a heart rate of a wearer of a biosensor based on photoplethysmogram (PPG) and acceleration data generated by the biosensor. The method can comprise: obtaining a first portion of the PPG data corresponding to PPG data over a first period of time; obtaining a first portion of the acceleration data corresponding to acceleration data over the first period of time; filtering the first portion of the PPG data and first portion of the acceleration data; calculating a frequency spectrum of the filtered first portion of the PPG data; calculating a frequency spectrum of the filtered first portion of the acceleration data; generating an interim heart rate estimate of the wearer during the first period of time, based at least in part on the frequency spectrum of the filtered first portion of the PPG data and the frequency spectrum of the filtered first portion of the acceleration data; comparing the interim heart rate estimate to an estimated heart rate from a previous period of time to generate a final heart rate estimate of the wearer during the first period of time; and generating an output indicative of the final heart rate estimate.

In any of the embodiments disclosed herein, filtering the first portion of the PPG and acceleration data can comprise filtering the first portion of the PPG and acceleration data with a first order bandpass Butterworth filter.

In any of the embodiments disclosed herein, calculating the frequency spectrum of the filtered first portion of the PPG data and calculating a frequency spectrum of the filtered first portion of the acceleration data can comprise using a sparse signal reconstruction method.

In any of the embodiments disclosed herein, generating the interim heart rate estimate can comprise determining whether a peak in the frequency spectrum of the first portion of the acceleration data is less than or greater than a first predetermined threshold.

In any of the embodiments disclosed herein, if the peak in the frequency spectrum of the first portion of the acceleration data is determined to be less than the first predetermined threshold, the interim estimated heart rate can correspond to a frequency of a peak in the frequency spectrum of the first portion of the PPG data having the largest magnitude.

In any of the embodiments disclosed herein, if the peak in the frequency spectrum of the first portion of the acceleration data is determined to be greater than the first predetermined threshold, the interim estimated heart rate can correspond to a frequency of a peak in the frequency spectrum of the first portion of the PPG data having a frequency closest to a final heart rate estimate from a previous period of time.

In any of the embodiments disclosed herein, comparing the interim heart rate estimate to an estimated heart rate from a previous period of time to generate a final heart rate estimate of the wearer during the first period of time can comprise: determining whether a magnitude of a difference between the interim estimated heart rate and the estimated heart rate from the previous period of time is less than or greater than a second predetermined threshold; if the difference between the interim estimated heart rate and the estimated heart rate from the previous period of time is less than the second predetermined threshold, setting the final heart rate estimate to the interim estimated heart rate; and if the difference between the interim estimated heart rate and the estimated heart rate from the previous period of time is greater than the second predetermined threshold, setting the final heart rate estimate to the estimated heart rate from the previous period of time.

Another embodiment of the present disclosure provides a system for estimating a heart rate of a wearer of a biosensor. The system can comprise any biosensor disclosed herein and a remote device. The remote device can comprise a transceiver, a processor, and a memory. The transceiver can be configured to receive the one or more signals indicative of a wearer's photoplethysmogram (PPG) and acceleration from the biosensor. The memory can comprise instructions that, when executed by the processor, cause the processor to implement any of the methods disclosed herein.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein.

Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIGS. 1A-D provide an overview certain aspects of a biosensor, in accordance with an exemplary embodiment of the present disclosure: FIG. 1A illustrates exemplary wireless monitoring of an athlete's health and performance with an exemplary biosensor (left), mounted on the wrist (right) showing a small, flexible form factor; FIG. 1B provides non-limiting examples of measured PPG (top) and acceleration (bottom) data from an exemplary biosensor;

FIG. 1C illustrates the multi-layered structure of an exemplary biosensor; and FIG. 1D provides a flow chart capturing data acquisition from an exemplary biosensor, signal processing, and sensor outputs.

FIGS. 2A-F provide data from a study of mechanical behavior and reliability of an biosensor in accordance with an exemplary embodiment of the present disclosure: FIG. 2A provides FEA result (left) of an exemplary biosensor upon 180° bending with radius of curvature of 1.5 mm (scale bar: maximum principal strain) and photos (right) showing mechanical flexibility of the exemplary biosensor; FIG. 2B provides measured electrical resistance of an exemplary biosensor upon cyclic bending (100 cycles) between 0 and 180°, showing negligible change; FIG. 2C provides photographs of an exemplary biosensor on the wrist under flowing water; FIG. 2D provides PPG data measured with flowing water, showing no adverse effect of water; FIG. 2E provides a comparison of thermal loading between a conventional rigid wristband device (left) and an exemplary biosensor (right) after one hour wearing; and FIG. 2F provides an RSSI response of an exemplary with and without a worn glove.

FIGS. 3A-D provide a comparison of device performance between a biosensor in accordance with an exemplary embodiment of the present disclosure and a conventional rigid wristband system: FIG. 3A provides photographs showing a conventional rigid-sensor embedded wristband (left) and thin-film based exemplary biosensor (right); FIG. 3B provides a comparison of PPG signals measured by the conventional rigid device with different levels of band tightness and an exemplary biosensor, along with calculated PPG RMS; FIG. 3C provides a photograph of a subject running on a treadmill wearing both a conventional rigid device and an exemplary biosensor; FIG. 3D provides a comparison of motion artifact data, extracted from tightly worn conventional rigid device and an exemplary biosensor (top) and loosely worn conventional rigid device and an exemplary biosensor (bottom), along with calculated noise RMS values.

FIGS. 4A-G provide a signal processing algorithm and measured data with a biosensor in accordance with an exemplary embodiment of the present disclosure: FIG. 4A provides a flowchart for identification of motion artifacts and heart rate (HR) estimation with sparse signal reconstruction (SSR); FIG. 4B provides bandpass filtered PPG data while standing still (left) and running on a treadmill (right); FIG. 4C provides acceleration data while standing still (left) and running on a treadmill (right); FIG. 4D provides frequency spectrum of PPG data via SSR, showing measured HR and motion artifact; FIG. 4E provides frequency spectrum of acceleration data via SSR, showing motion artifact; FIG. 4F provides a comparison of HR data, estimated by an exemplary biosensor and extracted from ECG; and FIG. 4G provides a HR data comparison when a subject runs for 5 minutes.

FIGS. 5A-D provide an analysis of impact velocity, impact force, and estimated force from acceleration of a biosensor in accordance with an exemplary embodiment of the present disclosure: FIG. 5A provides a photograph showing an experimental setup for measurement of impact force and impact velocity; FIG. 5B provides 3-axis acceleration data measured by an exemplary biosensor during a typical boxing punching; FIG. 5C provides a correlation of measured impact velocity from acceleration with measured impact force from punching (n=492); and FIG. 5D provides a comparison between measured impact force and estimated force using the linear fit in FIG. 5C of punches (n=212).

FIG. 6 provides a schematic illustration of microfabrication process of exemplary biosensor flexible circuitry, and its integration with electronic components and elastomer substrate in accordance with an exemplary embodiment of the present disclosure.

FIGS. 7A-B provide the structure and calibration of a force plate: FIG. 7A provides an exploded view illustration showing each layer of the force plate; and FIG. 7B provides a force plate calibration and its sensitivity in accordance with an exemplary embodiment of the present disclosure.

FIGS. 8A-B provide a top (FIG. 8A) and bottom (FIG. 8B) view illustration of flexible circuitry and electronic components of an exemplary biosensor with annotated functional blocks (a list of individual electronic components used for is set forth in Table 1), in accordance with an exemplary embodiment of the present disclosure.

FIG. 9 provides a battery voltage measurement during an exemplary biosensor battery life test, in accordance with an exemplary embodiment of the present disclosure.

FIGS. 10A-E provide a cyclic bending test of a biosensor in accordance with an exemplary embodiment of the present disclosure: FIGS. 10A-C provide photographs showing a sequence (0°, 90°, and 180°) of cyclic bending test of an exemplary biosensor with highly flexible circuitry (100 cycles), where FIG. 10A is at 0°, FIG. 10B is at 90°, and FIG. 10C is at 180°; FIG. 10D provides three-axis accelerometer data obtained during cyclic bending; FIG. 10E provides acceleration data of the first bending cycle; and FIG. 10E provides acceleration data of the last three bending cycles.

FIGS. 11A-B provides the measurement of adhesion strength between a biosensor in accordance with an exemplary embodiment of the present disclosure and skin: FIG. 11A provides a photograph showing a quantitative measurement of an adhesion strength between an exemplary biosensor and skin via a force-meter (MARK-10); and FIG. 11B provides the result of measured adhesion strength over the peel-off distance found by dividing total peel-force by the width of the substrate, and average adhesion strength at steady-state (0.0581 N/m).

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

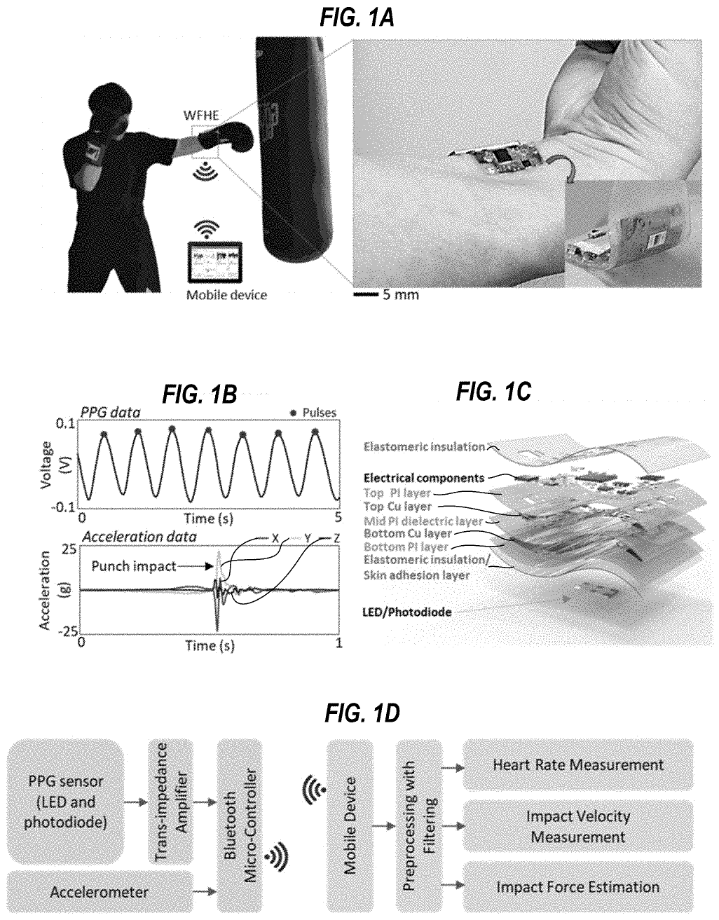

An exemplary embodiment of the present disclosure provides a wearable flexible biosensor. As shown in FIGS. 1A & 1B, the biosensor can be configured as a patch, which can be worn, among other places, on the wrist area of a wearer. The biosensor can comprise an electrical circuit made of a plurality of electrical components, including, but not limited to, electrical interconnects, microcontrollers, memory, transceivers, amplifiers, batteries, diodes, photodiodes, accelerometers, and the like. The electrical circuit can be configured to generate one or more signals indicative of a wearer's PPG and accelerations. For example, the electrical circuit can comprise an accelerometer used to generate a signal indicative of acceleration of the wearer. Additionally, the electrical circuit can comprise a photodiode and two LEDs (e.g., green LEDs) used to generate a signal indicative of the wearer's PPG. The photodiode and LEDs can be positioned proximate a bottom surface of the biosensor close to the skin of the user. In particular, the LEDs can direct light towards the skin of the wearer and the photodiode can receive light reflected from the wearer. The electrical circuit could further comprise a transceiver configured to transmit the signals to a remote device via, for example, Bluetooth technology.

The electrical circuit can be encapsulated by an elastomer. The elastomer can comprise many different elastomers known in the art, including, but not limited to, elastomers with a low-modulus like silicone. The elastomer can have a bottom surface configured to adhere to the skin of the wearer.

The elastomer can provide both flexibility and waterproofing characteristics to the biosensor. The flexibility of the biosensor can be useful in minimizing the effects of motion artifacts in the PPG signal on estimating heartrates of the user. In some embodiments, the biosensor can be capable of bending 180 degrees with a radius of curvature of about 1.0 mm to about 5.0 mm, about 1.0 mm to about 4.0 mm, about 1.0 mm to about 3.0 mm, about 1.0 mm to about 2.0 mm, about 1.25 mm to about 2.0 mm, about 1.25 mm to about 1.75 mm, or about 1.5 mm. The capability of the biosensor to withstand repeated bending can be shown through measured changes in resistance when the biosensor is subjected to cycled bending. For example, in some embodiments, the biosensor can be configured such that a resistance between an input and output of the electrical circuit changes less than 1.0 ohms if the biosensor is subjected to 100 cycles of bending over a range of 0 to 180 degrees with a minimum radius of curvature of 1.5 mm. In some embodiments, the biosensor can be configured such that a resistance between an input and output of the electrical circuit changes between 0.001 ohms and 1.0 ohms, between 0.001 ohms and 0.5 ohms, or between 0.001 ohms and 0.25 ohms, if the biosensor is subjected to 100 cycles of bending over a range of 0 to 180 degrees with a minimum radius of curvature of 1.5 mm.

The biosensor can comprise a battery configured to provide power to the biosensor. In some embodiments, the battery can be a rechargeable battery. In some embodiments, the battery can comprise charging terminals. In some embodiments, the charging terminals can be exposed from the elastomer such that they can contact electrodes of a battery charger. In some embodiments, however, the charging terminals can also be encapsulated by the elastomer and the battery can be recharged via magnetic charging.

As shown in FIG. 1C, the biosensor can comprise a plurality of layers. For example, the biosensor can comprise a first polymer layer (which can be many polymers, including, but not limited to polyimide and the like), a first electrical interconnect layer (which can be many electrically conductive materials, including, but not limited to copper), a dielectric layer, a second electrical interconnect layer (which can be many electrically conductive materials, including, but not limited to copper), and a second polymer layer (which can be many polymers, including, but not limited to polyimide and the like). The first polymer layer can be positioned beneath the electrical circuit. The first electrical interconnect layer can be positioned beneath the first polymer layer. The dielectric layer can be positioned beneath the first electrical interconnect layer. The second electrical interconnect layer can be positioned beneath the dielectric layer. The second polymer layer can be positioned beneath the second electrical interconnect layer. The elastomer can encapsulate the first polymer layer, the first electrical interconnect layer, the dielectric layer, the second electrical interconnect layer, and the second polymer layer. The electrical interconnect layers can be used to interconnect various components of the electrical circuit.

As discussed above, the biosensor can comprise a transceiver configured to transmit the PPG and acceleration signals to a remote device. The remote device can be any remote device known in the art, including, but not limited to, a desktop computer, a laptop computer, a smartphone, a tablet, and the like. The remote device can comprise a processor and memory. The memory can comprise instructions that, when executed by the processor, cause the remote device to perform a method to estimate a heartrate of the wearer. Processes for estimating the heart rate of the user are summarized above. Additionally, an exemplary method of estimating the heartrate of a wearer is described in the example below.

The following examples further illustrate aspects of the present disclosure. However, they are in no way a limitation of the teachings or disclosure of the present disclosure as set forth herein.

### EXAMPLES

Described below is an exemplary wireless, soft, wearable flexible hybrid electronic (WFHE) biosensor that measures HR from PPG signals, while analyzing a wearer's performance via 3-axis acceleration. This highly flexible device is constructed with multi-layered membranes, including copper and polyimide encapsulated by a soft elastomer. WFHE enables conformal, reliable lamination onto skin without the use of a wristband or strap. Comparison of PPG and HR data with a rigid wristband device and commercial system verifies WFHE's ability for reliable and continuous PPG and HR measurement. In addition, a study of impact velocity and force estimation process could be correlated and analyzed with the liberty of the miniaturized and ultra-thin nature of our device. Therefore, collective results indicate that this thin-film, flexible, and miniaturized wearable biosensor provides a novel solution in reliable, long-term health monitoring and athletic performance analysis while maintaining seamless skin contact.

Materials and Methods

Fabrication of WFHE

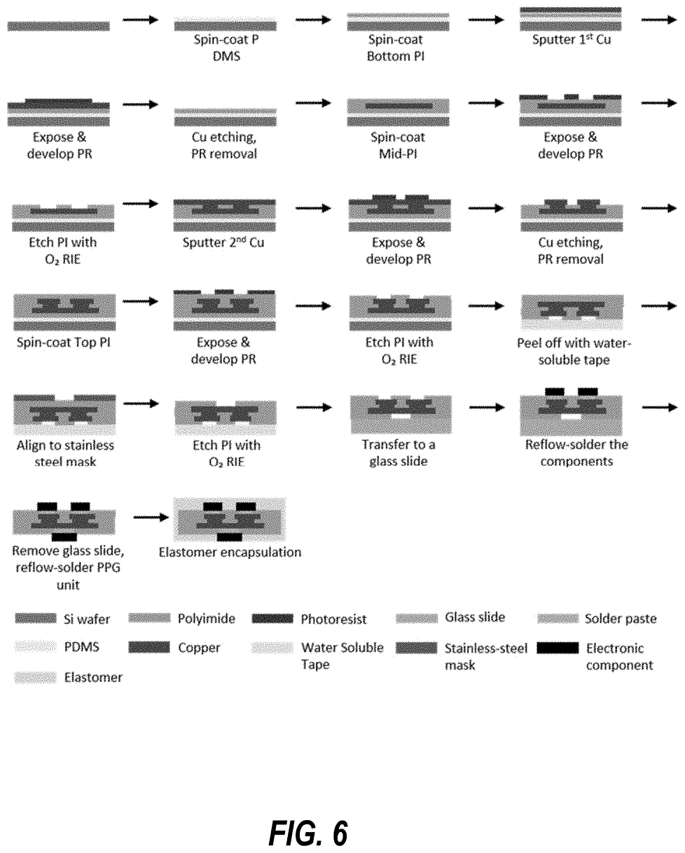

Fabrication of WFHE comprises two steps: (1) patterning of thin film-based electronic circuit by photolithography and other micromachining techniques; and (2) integration of functional chip components and soft material packaging. Details of the fabrication process are illustrated in FIG. 6 and described in detail below. Fabrication of a flexible circuit starts with a Si wafer coated with polydimethylsiloxane (PDMS). Polyimide (PI) is coated before a copper layer is deposited on the PDMS-coated wafer. Photolithography designs a multi-layered wireless circuit. The patterned circuit is peeled off the PDMS surface, and then functional chip components are soldered on the contact pads. Key sensors used in this device include a PPG sensor (SFH7070, Osram) and wide-range 6-axis inertial sensor (ICM-20649, InvenSense), which are integrated on the thin-film circuit by reflow soldering with a paste (SMDLTLFP10T5, Chip Quik). A small lithium polymer battery (LP401230, Adafruit) with a slide switch is connected to the device for easy use, while two small magnets are integrated on the battery-recharging pads for a convenient charging. The entire device, except the slide switch and opening of the PPG sensor, is fully encapsulated by a low-modulus, thin layer of a silicone elastomer (Ecoflex Gel, Smooth-On) for skin lamination.

Fabrication of the exemplary flexible circuit board was performed using the following process: (1) Spincoat PDMS (4:1 base-curing-agent ratio) on a silicon wafer at 4000 RPM for 30 seconds; (2) Treat PDMS surface with oxygen plasma at 200 W for 8 seconds; (3) Spincoat bottom polyimide layer (PI-2610, HD MicroSystems) at 2000 RPM for 60 seconds; (4) Soft bake at 100° C. for 5 minutes and hard bake at 250° C. for 1 hour; (5) Sputter 1.5 μm of Cu.; (6) Spincoat photoresist (PR, Microposit SC1813, MicroChem) at 3000 RPM for 30 seconds, and soft bake at 100° C. for 3 minutes; (7) Align with a photomask and expose UV light with intensity of 15 mJ/cm² for 10 seconds; (8) Develop UV-treated PR with a developer (MF-319, MicroChem); (9) Etch exposed Cu with Cu etchant (APS-100, Transene); (10) Remove remaining PR with acetone and foam swab, and rinse with IPA and DI water; (11) Spincoat 2^{nd} PI layer (PI-2545, HD MicroSystems) at 1000 RPM for 60 seconds, and soft bake at 100° C. for 10 minutes; (12) Spincoat additional 2^{nd} PI layer (PI-2545, HD MicroSystems) at 1000 RPM for 60 seconds, and soft bake at 100° C. for 10 minutes; (13) Hard bake at 250° C. for 2 hours in a vacuum oven; (14) Spincoat PR (AZ P4620, Integrated Micro Materials) at 900 RPM for 30 seconds, and soft bake at 90° C. for 5 minutes; (14) Align with a photomask and expose UV light with intensity of 15 mJ/cm² for 120 seconds; (15) Develop exposed PR with a developer (AZ-400K, Integrated Micro Materials) diluted with DI water (AZ400K:DI water=1:4); (16) Etch exposed PI with reactive ion etcher (RIE) at 200 W, 150 mTorr, and 20 sccm of oxygen for 40 minutes; (16) Remove remaining PR with acetone and foam swab, and rinse with IPA and DI water; (17) Sputter 1.5 μm of Cu; (18) Spincoat PR (AZ P4620, Integrated Micro Materials) at 900 RPM for 30 sec, and soft bake at 90° C. for 5 minutes; (19) Align with a photomask and expose UV light with intensity of 15 mJ/cm² for 120 seconds; (20) Develop exposed PR with a developer (AZ-400K, Integrated Micro Materials) diluted with DI water (AZ400K:DI water=1:4); (21) Etch exposed Cu with Cu etchant (APS-100, Transene); (22) Remove remaining PR with acetone and foam swab, and rinse with IPA and DI water; (23) Spincoat Top PI layer (PI-2545, HD MicroSystems) at 2000 RPM for 60 seconds; (24) Soft bake at 100° C. for 5 minutes and hard bake at 250° C. for 100 minutes in a vacuum oven; (25) Spincoat PR (AZ P4620, Integrated Micro Materials) at 2000 RPM for 30 seconds, and soft bake at 90° C. for 5 minutes; (26) Align with a photomask and expose UV light with intensity of 15 mJ/cm² for 90 seconds; (27) Develop exposed PR with a developer (AZ-400K, Integrated Micro Materials) diluted with DI water (AZ400K:DI water=1:4); (28) Etch exposed PI with reactive ion etcher (RIE) at 200 W, 150 mTorr, and 20 sccm of oxygen for 20 minutes; (29) Remove remaining PR with acetone and foam swab, and rinse with IPA and DI water; (30) Peel off the microfabricated circuitry with water-soluble tape; (31) Align the backside of circuitry to a stainless-steel mask for backside opening; and (32) Etch exposed PI with reactive ion etcher (RIE) at 200 W, 150 mTorr, and 20 sccm of oxygen for 20 minutes.

The flexible circuit board was integrated with the electronics components using the following process: (1) Transfer the flexible circuit board on a glass slide, and remove water soluble tape with DI water; (2) Screen-print low-temperature solder paste (alloy of Sn/Bi/Ag (42%/57.6%/0.4%), ChipQuik Inc.) with stainless-steel stencil on the top surface of circuitry; (3) Mount the electronics components; (4) Reflow solder the components on a hot plate by applying heat according to the temperature profile recommended by the solder paste manufacturer; (5) Update the firmware of the Bluetooth-microcontroller; and (6) Peel off from the glass slide, and solder LED/photodiode unit on the back side of the circuitry by locally applying heat with hot-air gun.

The circuit board with integrated electrical components were encapsulated with the elastomer using the following process: (1) Prepare 7.5 g of Ecoflex Gel (Smooth-On), and 2.5 g of Ecoflex 00-30 (Smooth-On) and mix them together to make 500 μm thick elastomer membrane; (2) Pour the elastomer mixture onto a polystyrene petri dish (FB0875714, Fisher Scientific); (3) Cure the elastomer at room temperature for 5 hours; (4) Cut and peel off the elastomer membrane from the petri dish; (5) Cut out a hole on the membrane where the PPG unit will be exposed; (6) Transfer the flexible circuit onto the elastomer membrane; and (7) Encapsulate the remaining exposed circuit area with Ecoflex 00-30.

Computational Mechanics with Finite Element Analysis

Finite element analysis to design WFHE in this work was conducted by using commercial software (Abaqus, Dassault Systemes). The focus of this study was on mechanical reliability of the device upon repetitive bending when mounted on the wrist. The modeling study estimated the maximum principal strain in multiple locations of joints that incorporate rigid chip components on membrane interconnects. The following material properties were used in the mechanics modeling study (E: Young's Modulus and v: Poisson's Ratio): E_{Cu}=119 GPa and v_{Cu}=0.34 for copper; E_{P1}=2.5 GPa and v_{P1}=0.34 for polyimide.

Experimental Study of the Device Reliability

Cyclic bending experiment (up to 100 cycles) of a fabricated WFHE was conducted to validate the result from computational modeling and mechanical reliability. The device was fixed to a pair of holders, allowing bending from 0 to 180° with a radius of curvature of 1.5 mm. A programmable motorized tester (ESM303, Mark-10) applied consistent bending cycles throughout the experiment. Thin copper wires (0.1 mm diameter) were connected to contact pads of the device to quantify the change of electrical resistance, which was measured by a digital multimeter (DMM7510, Keithley). With the same experimental setup, the quality of wirelessly transmitted data of motion activity during the cyclic bending were also measured. In addition to the mechanical test, the device's reliability in sweating and washing was measured by pouring water on the wrist worn WFHE, while monitoring wireless PPG signals for 5 minutes. Furthermore, considering a long, continuous use of the device during athletic training and matches, thermal loading on the skin was investigated. Infrared thermography (E8, FLIR) measured temperature profiles on the skin after wearing a device over an hour, which compared WFHE with a commercial rigid device. Lastly, a long-range, Bluetooth-based telemetry was studied by measuring a received signal strength indicator (RSSI) of WFHE. By mimicking a boxer, WFHE was worn on the wrist beneath a boxing glove, while measuring the signal quality up to 15 meters. The measured data with a glove was compared with the case on a bare wrist.

Acquisition of Health and Performance Data

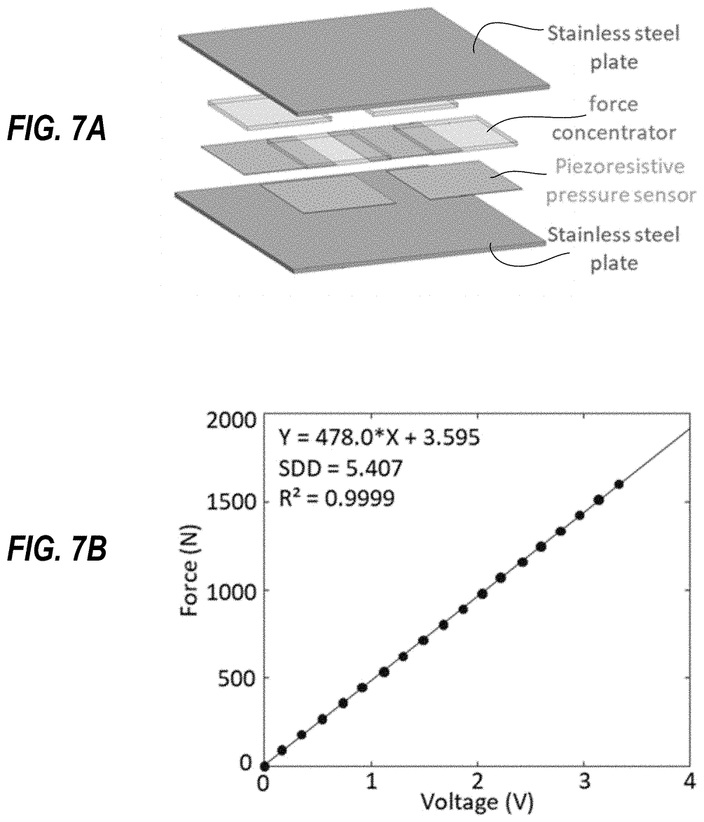

For PPG data collection, a subject wore both a wristband device and thin-film WFHE on the wrist. Since commercially available devices do not share the raw data, we customized a rigid-sensor device by integrating a custom PCB with a commercial wristband (Mi Band 2, Xiaomi). Note that the PCB circuit has the same functionality as the fabricated WFHE by sharing same chip components. With both devices, PPG was measured at the sampling rate of 100 Hz with multiple scenarios when the subject was sitting, walking, and running on a treadmill. These data were used to compare the effect of motion artifacts. In addition, HR was collected by a PPG sensor with two green LEDs and a photodiode, through a trans-impedance amplifier circuitry. A subject's motion activities were measured by a wide-range 6-axis inertial sensor (ICM-20649, InvenSense). For a punch impact force-velocity analysis, a subject had WFHE on the wrist with a boxing glove on top of the device. The custom-built force plate is composed of an array of four piezoresistive force sensors (FlexiForce A502 Sensor, Tekscan) sandwiched by two stainless steel plates and acrylic force concentrator on each sensor as shown in FIG. 7A. The force plate is calibrated with weights with known masses before the experiment, and its calibration plot is shown in FIG. 7B. Force sensor data was collected by a commercial data acquisition system (FlexiForce OEM Development Kit, Tekscan). The acceleration is measured at the sampling rate of 250 Hz, and the force is measured at 1000 Hz. During the punching experiment, WFHE on the wrist measured acceleration, while the plate recorded the impact force.

Data Acquisition with an Android Mobile Device

All of the physiological data from WFHE with Bluetooth communication were wirelessly monitored and recorded by an Android-tablet (Samsung Galaxy Tab) with a custom-designed application.

Study with Human Subjects

The study involved three healthy volunteers aged 18 to 40 and the study was conducted by following the approved Institutional Review Board protocol (#H17212) at Georgia Institute of Technology. The participating subjects signed consent forms and agreed to follow the given study protocols.

Results and Discussion

System Overview of WFHE

A wearable, flexible hybrid electronic system (WFHE) was developed. The WFHE has an exceptionally soft and small form factor with mechanical flexibility. The soft material-enabled, membrane system allows comfortable, seamless integration on the wrist, without the use of a rubber band or strap. This enables wireless, reliable monitoring of health and performance of athletes. FIG. 1 captures the overview of WFHE that has potentials to be used in athletic training, health, and performance assessment. Unlike the conventional wristband devices, WFHE is ultrathin, soft, and flexible, enabling unobtrusive lamination on the skin (FIG. 1A) with minimized mechanical and thermal loading. A soft elastomer (thickness: 1 mm, Ecoflex Gel, Smooth-On) that completely encapsulates WFHE provides sufficient adhesion for conformal lamination on the wrist, while avoiding mechanical slipping and dissociation of sensors. The small form factor allows a practical application of the device to wear athletic gears on hands and/or wrists. Compared to a commercial, flexible printed circuit board, the WFHE is about 10 times thinner, which results in a very small bending stiffness: 2 orders of magnitude smaller than a commercial flexible circuit. Details of the calculation and comparison of moment of inertia and bending stiffness are provided below. The skin-like membrane device has an embedded Bluetooth-low-energy unit on a mesh circuit to offer a long-range (up to 10 m), wireless detection of a subject's physiological data, including PPG and acceleration (FIG. 1B); these data are used to measure a real-time HR and impact force. Typically, electrocardiography (ECG) is considered as the gold-standard of HR measurement, but it requires multiple electrodes attached on various parts of human body. In contrast, PPG can be simply measured on a single, small area of the body, which could minimize the system's form factor. Besides the HR measurement, PPG can be further extended to detect blood pressure, respiration rate, and pulse oximetry.

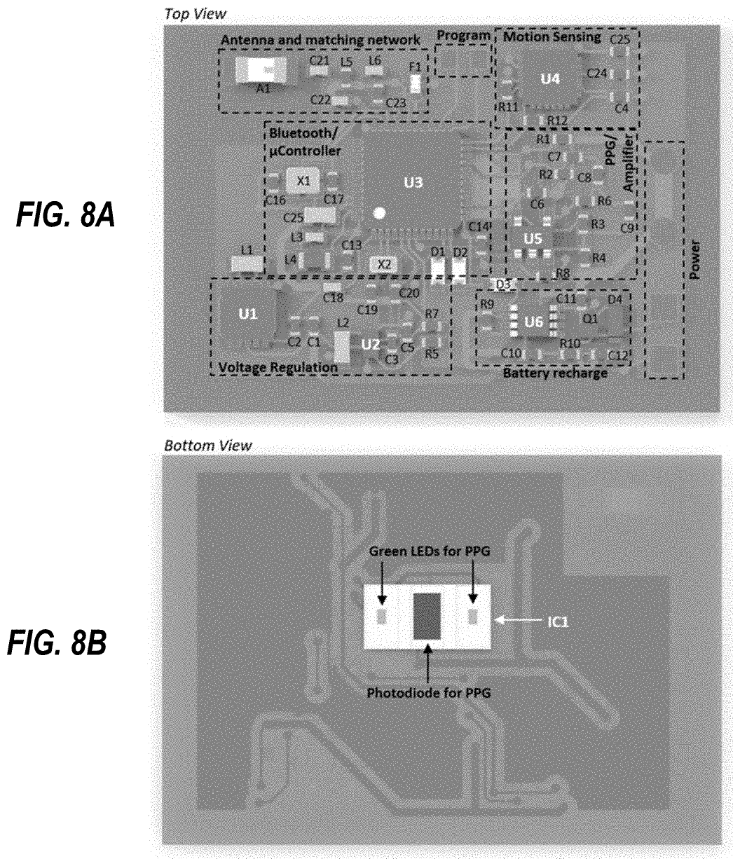

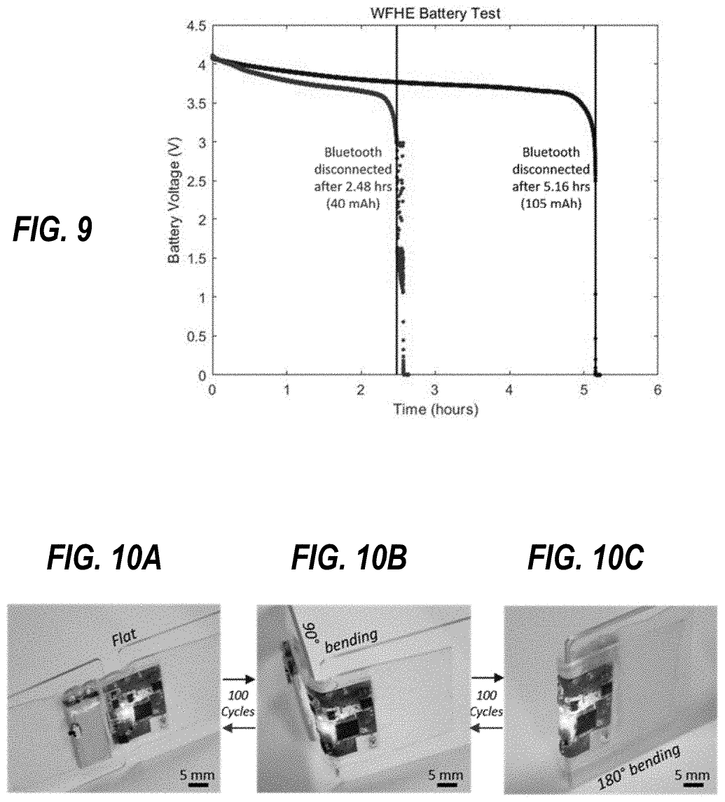

As shown in FIG. 1C, the wireless WFHE has a multi-layer structure, comprising a metal ground plane, dielectric PI layer, metal circuit interconnects, chip components, and elastomeric encapsulant. The top and bottom PI solder masks protect the copper (Cu) interconnects. All functional chips are directly integrated on the top Cu layer, except the PPG sensor (LED/photodiode) that has direct contact to the skin. Details of the flexible circuit design and enclosed components appear in Table 1 and FIG. 8. The device is powered by a rechargeable, lithium ion polymer battery (105 mAh, LP401230, Adafruit). A device operation time is around 5.16 hours with a continuous, simultaneous recording of PPG (sampling rate: 100 Hz) and acceleration data (sampling rate: 250 Hz). Results of the battery life test are shown in FIG. 9. After each use, the device can be easily recharged by attaching an external cable to small magnets on the circuit. A flow chart in FIG. 1D summarizes the entire process from signal acquisition to data transmission, and output analysis. PPG signals that are collected by a combination of two green LEDs and a photodiode go through a transimpedance amplifier to convert photodiode current to voltage output. The Bluetooth microcontroller collects and transmits both PPG and accelerometer data to an Android mobile device, where they are preprocessed with high-pass or band-pass filtering. Lastly, a data processing algorithm analyzes the measured signals to extract HR, movement velocity, and impact force of an athlete.

TABLE 1
List of electronic components for fabrication of WFHE.

| Component | Description | Value | | Part Number |
|---|---|---|---|---|
| U1 | 3.3 V voltage | N/A | | TPS63001 |
| | regulator | | | |
| U2 | 1.8 V voltage | N/A | | TPS62746 |
| | regulator | | | |
| U3 | Bluetooth PSoC | N/A | | NRF52832-QFAA-R |
| U4 | Motion sensor | N/A | | ICM20649 |
| U5 | Op Amp | N/A | | MCP6001 |
| U6 | Charge controller | N/A | | MCP73831 |
| IC1 | PPG sensor | N/A | | SFH7070 |
| A1 | 2.45 GHz RF chip | N/A | | 2450AT18A100 |
| | antenna | | | |
| F1 | 2.45 GHz low pass | N/A | | 2450FM07A0029 |
| | filter | | | |
| X1 | 32 MHz crystal | N/A | | ECS-320-8-37CKM |
| X2 | 32.768 kHz crystal | N/A | | ECS-327-9-12-TR |
| D1 | Blue LED | N/A | | LTST-C194TBKT |
| D2, D3 | Green LED | N/A | | APT1608ZGCK |
| D4 | Diode | N/A | | CDBU0530 |
| Q1 | MOSFET | N/A | | DMP21D5UFB4-78 |
| L1, L2 | 0603 inductor | 2.2 | μH | N/A |
| L3 | 0402 inductor | 15 | nH | N/A |
| L4 | 0603 inductor | 10 | μH | N/A |
| L5 | 0402 inductor | 10 | nH | N/A |
| L6 | 0402 inductor | 2.7 | nH | N/A |
| R1 | 0402 Resistor | 100 | Ω | N/A |
| R2, R6, R11, | 0402 Resistor | 10 | kΩ | N/A |
| R12 | | | | |
| R3, R4 | 0402 Resistor | 100 | kΩ | N/A |
| R5, R7 | 0402 Resistor | 1 | MΩ | N/A |
| R8 | 0402 Resistor | 10 | MΩ | N/A |
| R9 | 0402 Resistor | 2 | kΩ | N/A |
| R10 | 0402 Resistor | 13 | kΩ | N/A |
| C1, C3, C5 | 0402 capacitor | 10 | μF | N/A |
| C2 | 0402 capacitor | 22 | μF | N/A |
| C4, C14, | 0402 capacitor | 100 | nF | N/A |
| C18, C22, | | | | |
| C24, C25 | | | | |
| C6, C9 | 0402 capacitor | 2.2 | μF | N/A |
| C7, C8, C10, | 0402 capacitor | 4.7 | μF | N/A |
| C11, C13 | | | | |
| C12, C15 | 0402 capacitor | 1.0 | μF | N/A |
| C16, C17, | 0402 capacitor | 12 | pF | N/A |
| C19, C20 | | | | |
| C21 | 0402 capacitor | 0.4 | pF | N/A |
| C23 | 0402 capacitor | 100 | pF | N/A |

Calculation of Moment of Inertia and Bending Stiffness

To estimate and compare the moment of inertia and the resulting bending stiffness of WFHE and FPCB, each circuitry geometry is analytically modeled as a composite beam with one end fixed. Each layer is assumed to be a bulk material without any pattern. Force applied on the circuit by skin adhesion is modeled as a uniformly distributed load q. Bending stiffness of a beam under uniformly distributed load can be modeled as:

$$k = \frac{q L}{δ} = \frac{8 EI}{{L}^{3}}$$

Here, k is bending stiffness, q is distributed load, L is length of the circuit, δ is deflection of beam's tip, E is Young's modulus, and I is moment of inertia. Since circuit is treated as a composite beam here, transformed-section method is used to transform the cross-section of a composite beam into an equivalent cross-section of an imaginary beam that is composed of only one material. Width of each layer is determined as below:

$${w}_{2} = \frac{{E}_{2}}{{E}_{1}} {w}_{1}$$

Here, w₂ is the width of the layer that is being transformed, and w₁ is the width of the layer of a material that is used as material of transformed sections: polyimide (PI) in this analysis. The layers that are transformed are copper (Cu) layers. Moment of inertia of the transformed cross-section of the beam can be found using parallel-axis theorem, described as:

$${I}_{total} = \sum ( {\overline{I}}_{i} + {A}_{i} {d}_{i}^{2} ) where , \\ {\overline{I}}_{i} = \frac{1}{12} \frac{{E}_{i}}{{E}_{PI}} {w}_{i} {h}_{i}^{3}$$

$${A}_{i} = \frac{{E}_{i}}{{E}_{PI}} {w}_{i} {h}_{i}$$

$${d}_{i} = \vert {y}_{i} - \overline{y} \vert Therefore , \\ {I}_{total} = \sum \frac{{E}_{i}}{{E}_{PI}} {w}_{i} {h}_{i} ( \frac{1}{12} {h}_{i}^{2} + \vert {y}_{i} - \overline{y} \vert )$$

Here, Iₜₒₜₐₗ is the moment of inertia of entire cross-section, Iᵢ is moment of inertia of the individual layer about its own centroid axis, Aᵢ is the area of the individual layer, dᵢ is the vertical distance from the centroid of the layer to the neutral axis located at the centroid, hᵢ is thickness of individual layer, yᵢ is height of the centroid of each layer, and y̅ is neutral axis of cross-section. Substituting moment of inertia equation into bending stiffness equation yields:

$$k = \frac{8 {E}_{PI}}{{L}^{3}} \sum \frac{{E}_{i}}{{E}_{PI}} {w}_{i} {h}_{i} ( \frac{1}{12} {h}_{i}^{2} + \vert {y}_{i} - \overline{y} \vert )$$

Both WFHE and FPCB are composed of three PI layers and two Cu layers as illustrated in FIG. 1C. In the present study, thickness of each layer is WFHE is 2.6 μm, 1.5 μm, 8.7 μm, 1.5 μm, and 2.8 μm, for bottom PI, bottom Cu, mid PI, top Cu, and top PI layer, respectively. For FPCB, thickness profiles provided by the manufacturer (Shenzhen JDB Technology, Shenzhen, China) are used: 27.5 μm, 18 μm, 38.5 μm, 18 μm, and 27.5 μm, bottom PI, bottom Cu, mid PI, top Cu, and top PI layer, respectively. E_{P1} and E_{Cu} are 2.5 GPa and 119 GPa, respectively. The width (w) of the circuit is 19.36 mm, and the length (L) is 27.08 mm. Finally, the resulting bending stiffness of WFHE is 0.07954 N/m, and the bending stiffness of FPCB is 30.51 N/m.

Study of Mechanical Behavior and Reliability of WFHE

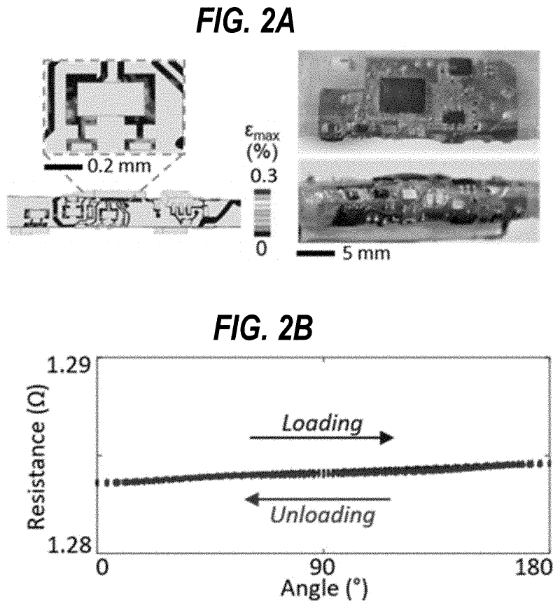

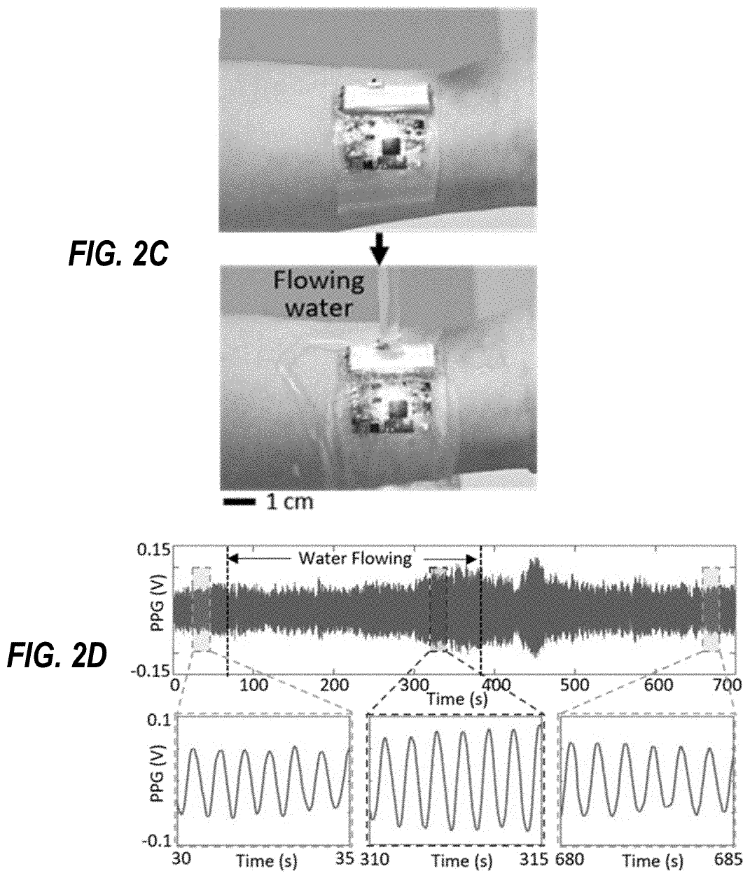

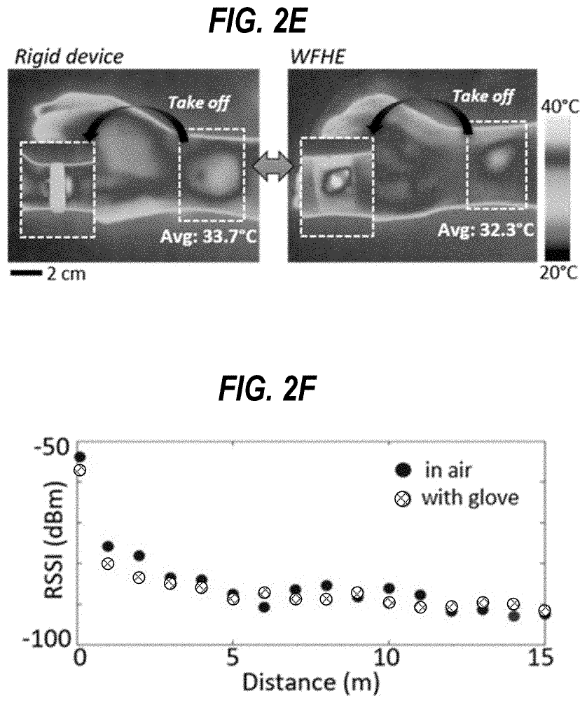

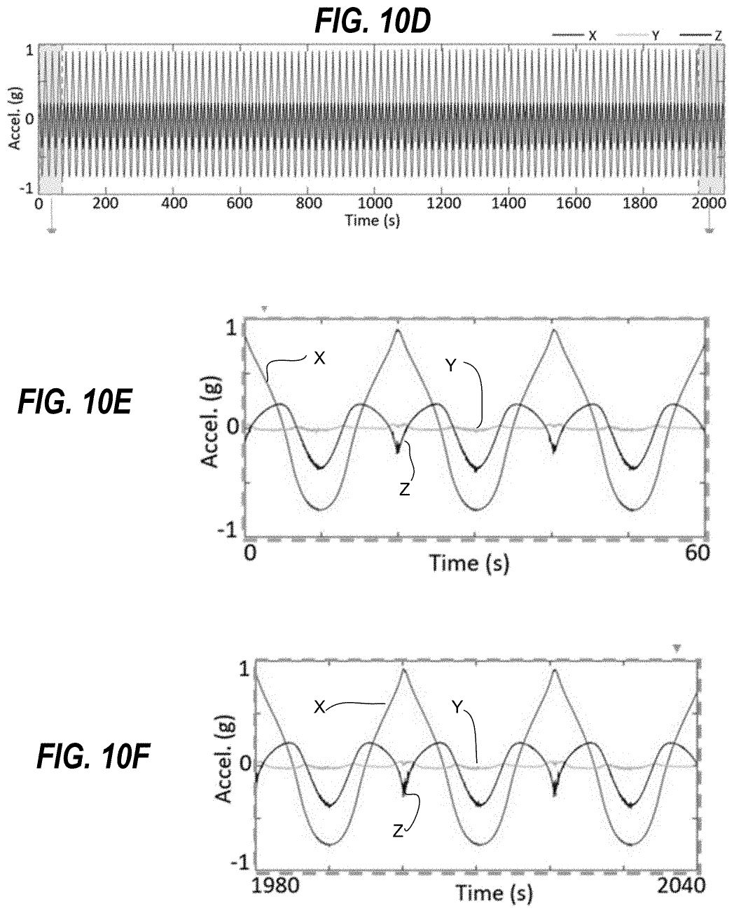

This study summarizes a set of computational analysis and experimental investigation to capture the device feasibility for practical use in athletic health and performance assessment. Prior to device fabrication, a computational mechanics modeling with 3D finite element analysis (FEA) was conducted to estimate the maximum principal strain of WFHE structure. Simulated 180° bending of WFHE with radius of curvature of 1.5 mm shows the maximum principal strain value (left, FIG. 2A), well under the yielding (0.3%) and fracture strain (5%) of Cu. An experimental bending test (right, FIG. 2A) validates mechanical safety of WFHE upon excessive bending, which is further quantified by measuring the change of electrical resistance of the device (FIG. 2B). The result from 100-cycle bending shows a minimal change of resistance (0.0013Ω), which supports the device's reliability. Wirelessly transmitted acceleration measured during a cyclic bending (FIG. 10), captures the device's consistent functionality, which also shows the flexibility and mechanical reliability of the WFHE.

In addition, another set of experiments, shown in FIGS. 2C & 2D, highlights the waterproof characteristic of the device. By mimicking excessive sweating during athletic training, flowing water was poured on the WFHE for 5 minutes (FIG. 2C). During the test, PPG signals were continuously monitored by a portable tablet that analyzes signal quality during both “dry” and “wet” periods (FIG. 2D), showing negligible effects of flowing water in the device performance. Furthermore, thermal loading of the wearable device on the skin was investigated by infrared thermography that compared WFHE with a rigid wristband device. After 1 hour of wearing the wrist device, change of temperature is compared as shown in FIG. 2E. The result shows that the bulky form factor of a rigid PCB and wristband causes accumulated heat on a large area of wrist with an average temperature of 33.7° C., while the membrane-based WFHE, with facilitated heat dissipation through its thin profile, shows lower temperature (32.3° C.) with a minimized heat spot. Another advantage of WFHE is in reliable wireless data acquisition even with a glove, worn on top of the device (FIG. 2F). The result of RSSI, measured with WFHE under a boxing glove, shows consistent signals, comparable to the device only case, at distances up to 15 meters. Overall, WFHE shows a great potential to measure physiological data with minimized mechanical and thermal loading, while still offering a seamless integration with athletic gears.

Comparison of Device Performance Between WFHE and Rigid Wristband System

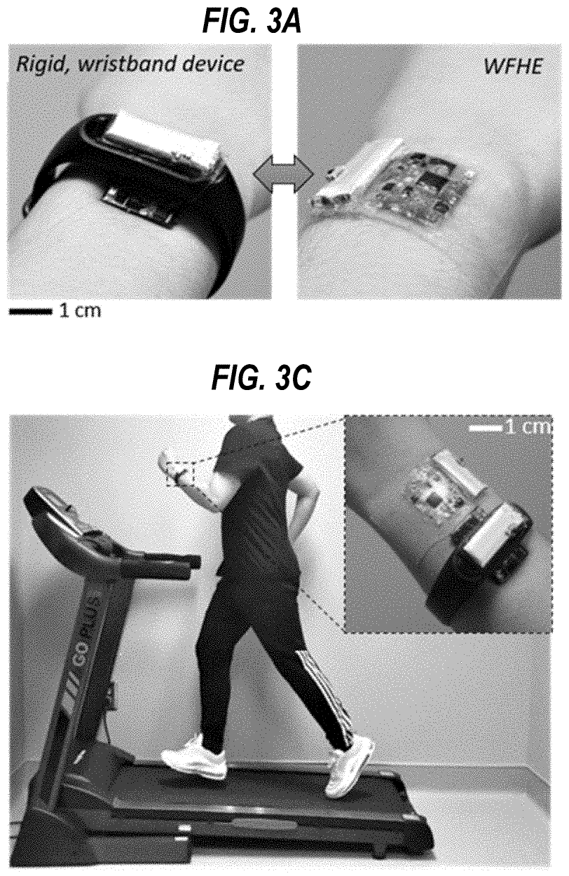

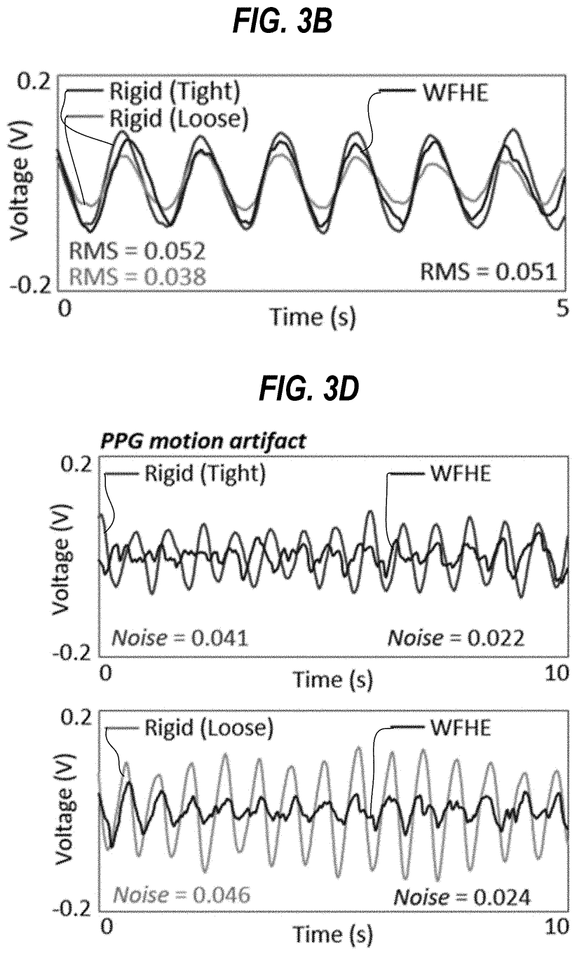

Conventional wearable devices that measure PPG, HR, and/or acceleration use rigid electronics, wrapped by a rubber band to wear on the wrist. Significant issues of the wristband-type devices are in signal loss and degradation, caused by the device slipping and dissociation. Here, a wristband device that embeds a wireless circuit was custom-designed, same as WFHE for a simultaneous comparison of signal quality. To compare performance between the rigid and flexible devices, a subject wore the two different wrist devices (FIG. 3A) to measure PPG signals. A portable tablet with a custom-made application monitors PPG data, recorded sequentially on the same location and the root-mean-square (RMS) voltage is calculated. The result in FIG. 3B shows that data quality from WFHE (V_{RMS}=0.051) is comparable to that from the rigid device (V_{RMS}=0.052) when the wristband has a very tight fit to the skin. However, the same rigid device loses a significant amount of signals when the subject has a loose fit (V_{RMS}=0.038). Although tight wearing of the rigid device with higher pressure on the skin offers good signals, this arrangement causes huge discomfort and movement disruption to the wearer. In this study, it was found that PPG signals, measured on the curved sides of inner wrist, have higher amplitude that those from outer wrist surface since higher volume of blood flow occurs through ulnar and radial arteries. Unlike the soft, highly deformable WFHE, the rigid device is prevented from maintaining continuous, proper contact on the curved skin surface due to the rigid electronics.

In addition, the rigid, wristband devices are typically susceptible and vulnerable to motion artifacts from diverse activities. Dealing with motion artifact has been an enormous challenge in HR estimation since frequency range between motion and PPG. Artifact signals contaminate the data and causes extensive and complex signal processing and analysis. To validate the advantage of a light, skin-conformal device, baseline noise was measured from a subject wearing two devices while running on a treadmill at a speed of 5 mph (FIG. 3C). PPG noise from motion artifact for the rigid device shows RMS amplitude of 0.041 V, which is nearly twice that from WFHE (0.021 V) (top graph in FIG. 3D). When the wristband device is worn with more comfortable loose fit, then the measured PPG noise increases (0.0461 V) due to continuous loss of the device contact on the wrist (bottom graph in FIG. 3D). Real-time PPG and acceleration data from both devices when a subject is running also shows a minimized effect of PPG motion artifacts on the WFHE compared to the rigid device. Thus, the skin-conformal WFHE is a more practical solution to continuously measure high quality HR signals of athletes during excessive training.

Development of a Signal Processing Algorithm

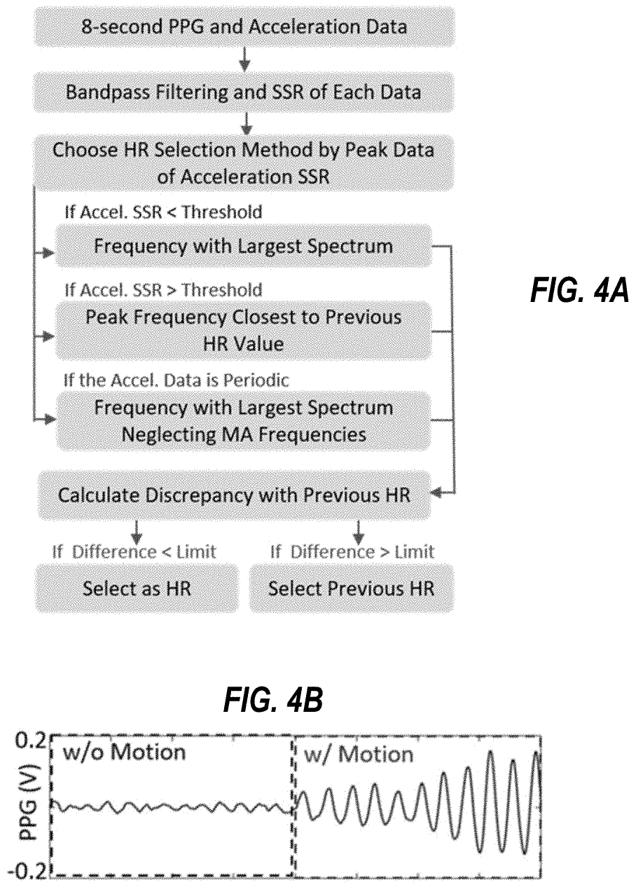

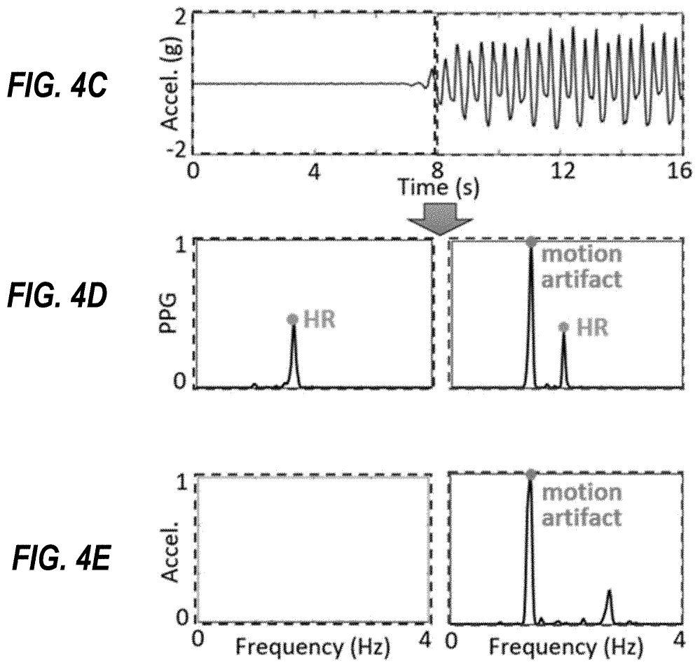

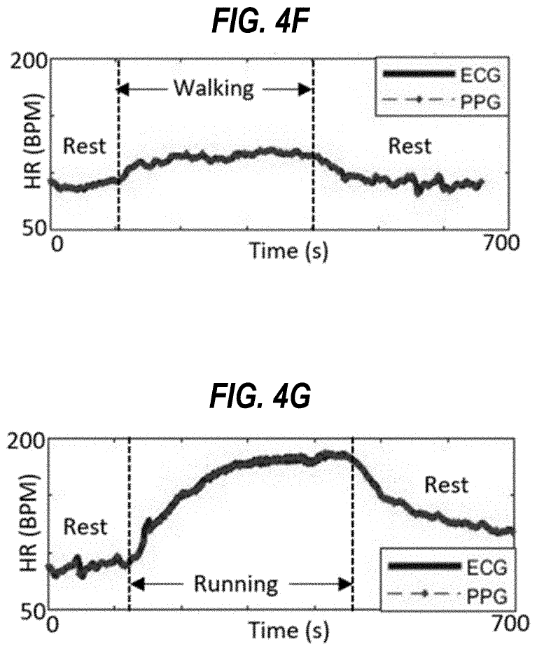

The functionality of WFHE was validated by developing a signal processing algorithm of PPG data. To detect HR from PPG data with motion artifact, the integrated accelerometer in WFHE is used to identify the frequency distribution of the motion artifact and PPG based on a spectral subtraction method. A flowchart in FIG. 4A describes the exemplary sequential process of HR detection with sparse signal reconstruction (SSR). To simulate the real-time estimation of HR, PPG and acceleration data are divided into 8-second segments and analyzed sequentially. Both data are preprocessed with a Pt order bandpass Butterworth filter at cutoff frequencies of 0.5 and 5 Hz. This process removes extraneous noise besides the range of frequencies in which the actual HR frequency presents (FIGS. 4B & 4C). Afterwards, an SSR method is used to calculate frequency spectrum of the preprocessed data (FIGS. 4D & 4E), which provides higher spectrum resolution with increased robustness compared to a conventional spectral estimation method such as Periodogram. Depending on the peak spectrum of the acceleration data, it is determined whether the data is severely affected by motion artifact (boxes in FIG. 4E). If the peak spectrum is less than the set threshold (left, FIG. 4E), the motion artifact is not considered to be severe. Then, HR is estimated to be the frequency corresponding to the peak frequency of the PPG spectrum (left, FIG. 4D). On the other hand, when the peak spectrum is above the set threshold, it is determined whether the activity is aperiodic or periodic. A short, impulsive accelerometer data is generated from an aperiodic motion such as hitting an object. Frequency spectrum of this data shows either one or multiple peaks above the threshold depending on the strength of the impact, which causes a short motion artifact in PPG data. In this case, peak spectrum of PPG is selected if there is just one peak above the set threshold. If there is more than one peak above PPG spectrum caused by a rapid motion, then the peak that is close to HR value determined from the previous data segment is selected. As an example, acceleration data from a periodic motion, such as walking or running, forms two clear peaks above the threshold (right, FIG. 4E), which causes continuous, periodic motion artifact in PPG signals, forming a single clear peak at the corresponding frequency of the motion artifact (right, FIG. 4D).

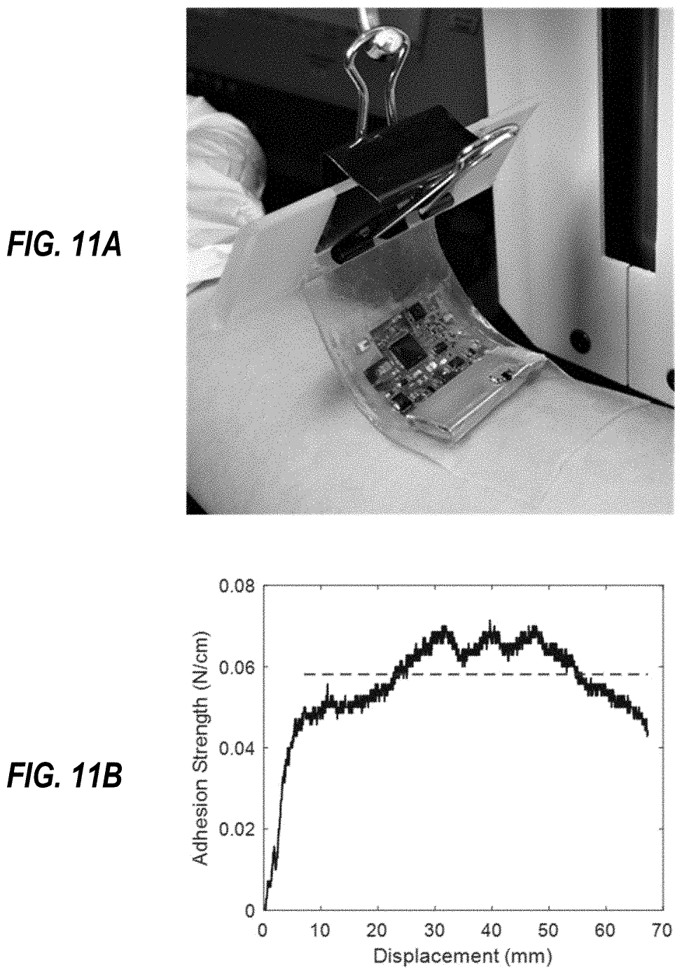

To evaluate the accuracy of the continuous HR estimation method, a simultaneous data comparison was conducted between WFHE and a clinical grade commercial device (BioRadio, Great Lakes NeuroTechnologies). We extracted HR data from measured ECG via the BioRadio (sampling rate: 250 Hz) by using the Pan-Tompkins algorithm. A subject, wearing both WFHE and BioRadio, was asked to walk at 3 mph and run at 6 mph for 5 minutes. The summarized results in FIG. 4F (walking) and FIG. 4G (running) show that HR values from WFHE has a great agreement with that from the ECG measurement. The averaged absolute difference between two datasets is only 0.59 BPM and 0.89 BPM for the case of walking and running, respectively. Overall, this result shows 55% higher accuracy than the average of absolute errors (2 BPM) from conventional systems. HR data could be accurately measured before, during, and after the activity, showing reliable contact of the WFHE with the skin due to the sufficient adhesion. To validate, an average adhesion strength between the device and the skin was measured by a digital peel-force test. As summarized in FIG. 11, the measured value was 0.0581 N/m, which falls into the similar range of the substrate's conformal contact, reported by the prior work. There is a possibility that a prolonged, excessive sweating, combined with air permeation, would diminish the adhesion.

Analysis of Impact Velocity and Force from Acceleration Data

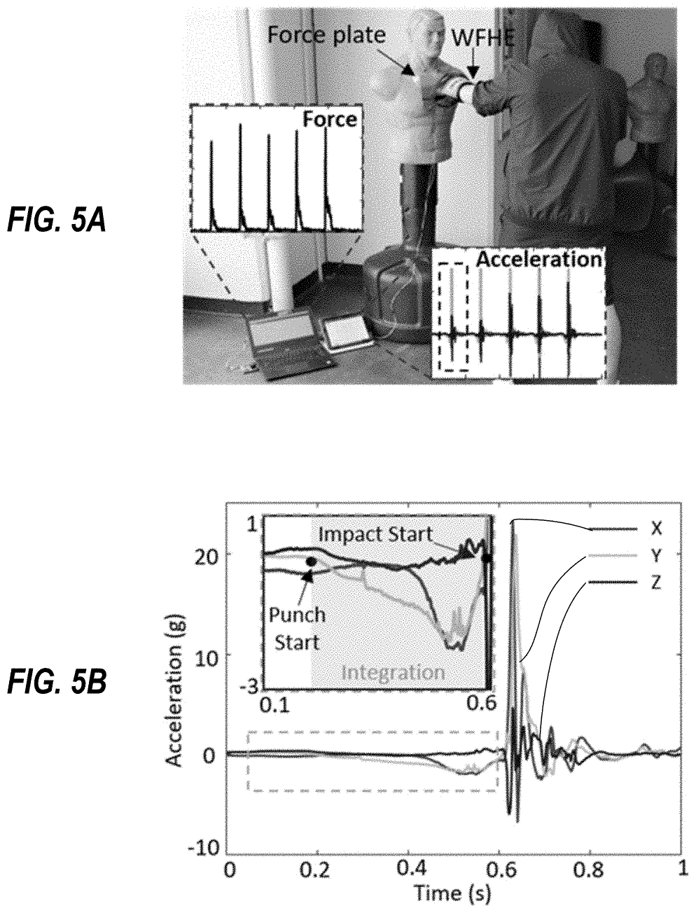

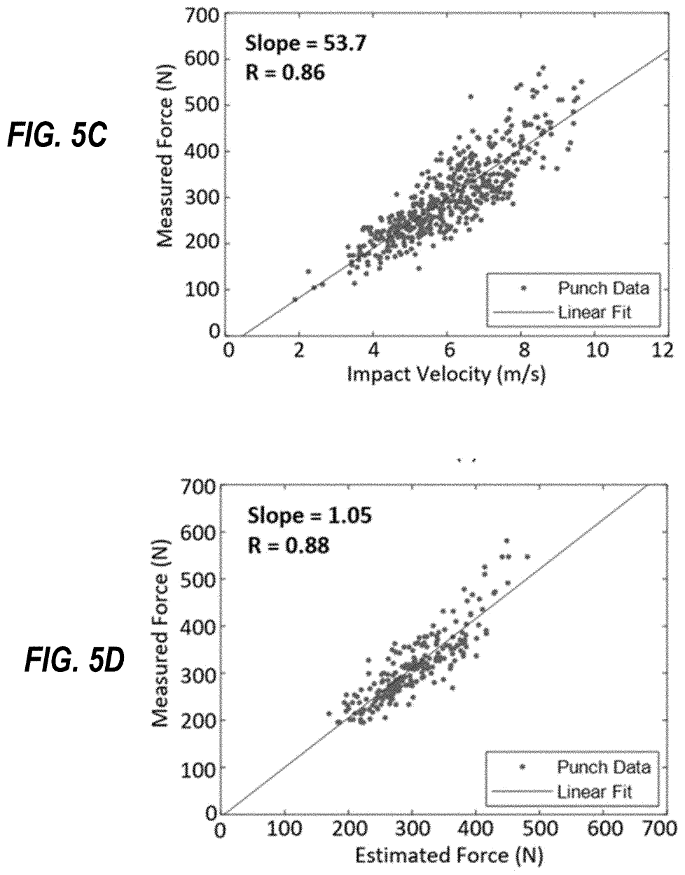

In order to observe an athletic performance, WFHE mounted on the wrist was used to evaluate punching, which would find potential applications in various combat sports, such as kickboxing, mixed martial arts, and boxing. According to prior research, impact force of punches by boxers most closely correlates with the impact velocity of the punches rather than other biomechanical factors such as sum of lower body forces and body weight. In this study, impact force and acceleration profile of straight punches of boxing are measured simultaneously with a custom-designed force plate and WFHE's accelerometer (FIG. 5A). The estimated impact velocity and measured forces are correlated for impact force estimation without direct force measurement. FIG. 5B shows a typical acceleration profile of a punch thrown in the Y-axis direction, with the largest peak corresponding to the point of impact. From the video recording of the experiment, it is determined that punches from three subjects start at about 0.4 seconds before the impact on average. Therefore, to estimate the impact velocity from this data, the acceleration data is integrated with trapezoidal rule from the point 0.4 seconds before the impact point. Then these velocity values are plotted with the measured peak impact forces of each punch to derive and evaluate linear regression and strength (FIG. 5C). The correlation coefficient value of 0.86 and standard uncertainty value of 45.51 N show a strong positive relationship between the impact velocity and force of the straight punches. From the second dataset measured in the same manner, impact forces are estimated with the linear regression found in the original dataset, and compared with the measured impact force (FIG. 5D). The result shows that the estimated and measured impact forces are well matched with the linear regression slope of 1.05 and the correlation coefficient of 0.876 with standard uncertainty value of 35.89 N.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A wearable flexible biosensor, comprising:
an electrical circuit configured to generate one or more signals indicative of a wearer's photoplethysmogram (PPG) and acceleration; and
an elastomer encapsulating the electrical circuit, the elastomer having a bottom surface configured to adhere to the skin of the wearer.

2. The wearable flexible biosensor of claim 1, further comprising:
at least two light emitting diodes proximate the bottom surface of the elastomer and configured to direct light towards the skin of the wearer; and
a photodiode proximate the bottom surface of the elastomer and configured to receive light reflected from the wearer.

3. The wearable flexible biosensor of claim 1, further comprising a rechargeable battery configured to provide power to the electrical circuit.

4. The wearable flexible biosensor of claim 1, further comprising:
a first polymer layer positioned beneath the electrical circuit;
a first copper electrical interconnect layer positioned beneath the first polymer layer;
a dielectric layer positioned beneath the first copper electrical interconnect layer;
a second copper electrical interconnect layer positioned beneath the dielectric layer; and
a second polymer layer positioned beneath the second copper electrical interconnect layer,
wherein the elastomer encapsulates the first polymer layer, the first copper electrical interconnect layer, the first copper electrical interconnect layer, the dielectric layer, the second copper electrical interconnect layer, and the second polymer layer.

5. The wearable flexible biosensor of claim 1, further comprising a wireless transceiver configured to transmit the one or more signals indicative of a wearer's photoplethysmogram (PPG) and acceleration to a remote device.

6. The wearable flexible biosensor of claim 1, wherein the elastomer is configured to prevent water exterior to the biosensor from migrating into electrical circuit.

7. The wearable flexible biosensor of claim 1, wherein the biosensor is capable of bending 180 degrees with a radius of curvature of about 1.5 mm.

8. The wearable flexible biosensor of claim 7, wherein the electrical circuit comprises an input and an output, wherein the biosensor is configured such that a resistance between the input and output changes less than 1.0 ohms if the biosensor is subjected to 100 cycles of bending over a range of 0 to 180 degrees with a minimum radius of curvature of 1.5 mm.

9. The wearable flexible biosensor of claim 8, wherein the electrical circuit comprises an input and an output, wherein the biosensor is configured such that a resistance between the input and output changes less than 0.001-0.5 ohms if the biosensor is subjected to 100 cycles of bending over a range of 0 to 180 degrees with a minimum radius of curvature of 1.5 mm.

10. The wearable flexible biosensor of claim 8, wherein the biosensor is configured as a patch.

11. A method of estimating a heart rate of a wearer of a biosensor based on photoplethysmogram (PPG) and acceleration data generated by the biosensor, comprising:
obtaining a first portion of the PPG data corresponding to PPG data over a first period of time;
obtaining a first portion of the acceleration data corresponding to acceleration data over the first period of time;
filtering the first portion of the PPG data and first portion of the acceleration data;
calculating a frequency spectrum of the filtered first portion of the PPG data;
calculating a frequency spectrum of the filtered first portion of the acceleration data;
generating an interim heart rate estimate of the wearer during the first period of time, based at least in part on the frequency spectrum of the filtered first portion of the PPG data and the frequency spectrum of the filtered first portion of the acceleration data;
comparing the interim heart rate estimate to an estimated heart rate from a previous period of time to generate a final heart rate estimate of the wearer during the first period of time; and
generating an output indicative of the final heart rate estimate.

12. The method of claim 11, wherein filtering the first portion of the PPG and acceleration data comprises filtering the first portion of the PPG and acceleration data with a first order bandpass Butterworth filter.

13. The method of claim 11, wherein calculating the frequency spectrum of the filtered first portion of the PPG data and calculating a frequency spectrum of the filtered first portion of the acceleration data comprises using a sparse signal reconstruction method.

14. The method of claim 11, wherein generating the interim heart rate estimate comprises determining whether a peak in the frequency spectrum of the first portion of the acceleration data is less than or greater than a first predetermined threshold.

15. The method of claim 14, wherein if the peak in the frequency spectrum of the first portion of the acceleration data is determined to be less than the first predetermined threshold, the interim estimated heart rate corresponds to a frequency of a peak in the frequency spectrum of the first portion of the PPG data having the largest magnitude.

16. The method of claim 14, wherein if the peak in the frequency spectrum of the first portion of the acceleration data is determined to be greater than the first predetermined threshold, the interim estimated heart rate corresponds to a frequency of a peak in the frequency spectrum of the first portion of the PPG data having a frequency closest to a final heart rate estimate from a previous period of time.

17. The method of claim 11, wherein comparing the interim heart rate estimate to an estimated heart rate from a previous period of time to generate a final heart rate estimate of the wearer during the first period of time, comprises:
determining whether a magnitude of a difference between the interim estimated heart rate and the estimated heart rate from the previous period of time is less than or greater than a second predetermined threshold;
if the difference between the interim estimated heart rate and the estimated heart rate from the previous period of time is less than the second predetermined threshold, setting the final heart rate estimate to the interim estimated heart rate; and
if the difference between the interim estimated heart rate and the estimated heart rate from the previous period of time is greater than the second predetermined threshold, setting the final heart rate estimate to the estimated heart rate from the previous period of time.

18. A system for estimating a heart rate of a wearer of a biosensor, the system comprising:
the biosensor of claim 1; and
a remote device comprising:
a transceiver configured to receive the one or more signals indicative of a wearer's photoplethysmogram (PPG) and acceleration from the biosensor;
a processor; and
a memory, the memory comprising instructions that, when executed by the processor, cause the processor to implement the method of claim 11.

19. The system of claim 18, wherein the memory comprises instructions that, when executed by the processor, cause the processor to implement the method of claim 15.

20. The system of claim 18, wherein the memory comprises instructions that, when executed by the processor, cause the processor to implement the method of claim 16.
